(19) United States
(12) Patent Application Publication
(10) Pub. No.: US 2011/0046504 A1
(43) Pub. Date: Feb. 24, 2011

(54) DISTRIBUTED NEURO-RESPONSE DATA COLLECTION AND ANALYSIS

(75) Inventors: Anantha Pradeep, Berkeley, CA (US); Robert T. Knight, Berkeley, CA (US); Ramachandran Gurumoorthy, Berkeley, CA (US)

Correspondence Address: Weaver Austin Villeneuve & Sampson LLP, P.O. BOX 70250, OAKLAND, CA, 94612-0250, US

(73) Assignee: NEUROFOCUS, INC., Berkeley, CA (US)

(21) Appl. No.: 12/846,242

(22) Filed: Jul. 29, 2010

Related U.S. Application Data
(63) Continuation of application No. 12/544,958, filed on Aug. 20, 2009.

(51) Int. Cl. A61B 5/0476; A61B 5/0496

(52) U.S. Cl. 600/544; 600/546

(57) ABSTRACT

Distributed mechanisms are provided for collecting neuro-response data from subjects exposed to the stimulus material in multiple settings. Stimulus material may include marketing and entertainment materials. Neuro-response data collection mechanisms such as Electroencephalography (EEG) and Electrooculography (EOG) are used to collect data from subjects in laboratory and corporate settings. Neuro-response data is transmitted over a network to a data analyzer. The neuro-response data is processed at the data analyzer and effectiveness data for the stimulus material is received.

### CROSS-REFERENCE TO RELATED APPLICATION

This application is a continuation of and claims priority to U.S. patent application Ser. No. 12/544,958 (Atty. Docket No. NFCSP029), filed Aug. 20, 2009, titled “DISTRIBUTED NEURO-RESPONSE DATA COLLECTION AND ANALYSIS,” all of which is incorporated herein by this reference for all purposes.

### TECHNICAL FIELD

The present disclosure relates to distributed neuro-response data collection and analysis.

### DESCRIPTION OF RELATED ART

Conventional systems for performing marketing and entertainment analysis typically involve monitoring and surveying individuals exposed to materials such as advertisements, programs, and commercials. Attempts have been made to allow a user to respond to user surveys quickly after viewing programs and commercials, but information collected is typically limited.

Consequently, it is desirable to provide improved mechanisms for performing distributed neuro-response data collection and analysis.

### BRIEF DESCRIPTION OF THE DRAWINGS

The disclosure may best be understood by reference to the following description taken in conjunction with the accompanying drawings, which illustrate particular example embodiments.

FIG. 1 illustrates one example of a system for performing distributed neuro-response data collection and analysis.

FIG. 2 illustrates examples of stimulus attributes that can be included in a stimulus attributes repository.

FIG. 3 illustrates examples of data models that can be used with a stimulus and response repository.

FIG. 4 illustrates one example of a query that can be used with the distributed neuro-response collection system.

FIG. 5 illustrates one example of a report generated using the distributed neuro-response collection system.

FIG. 6 illustrates one example of a technique for performing distributed neuro-response collection and analysis.

FIG. 7 provides one example of a system that can be used to implement one or more mechanisms.

### DESCRIPTION OF PARTICULAR EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques and mechanisms of the present invention will be described in the context of particular types of neuro-response data such as central nervous system, autonomic nervous system, and effector data. However, it should be noted that the techniques and mechanisms of the present invention apply to a variety of different types of data. It should be noted that various mechanisms and techniques can be applied to any type of stimuli. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

### OVERVIEW

Distributed mechanisms are provided for collecting neuro-response data from subjects exposed to the stimulus material in multiple settings. Stimulus material may include marketing and entertainment materials. Neuro-response data collection mechanisms such as Electroencephalography (EEG) and Electrooculography (EOG) are used to collect data from subjects in laboratory and corporate settings. Neuro-response data is transmitted over a network to a data analyzer. The neuro-response data is processed at the data analyzer and effectiveness data for the stimulus material is received.

### EXAMPLE EMBODIMENTS

Conventional distributed response monitoring mechanisms merely track stimulus being viewed and rely on behavior and survey based data collected from subjects exposed to marketing materials. In some instances, attempts are made to measure responses to programs and commercials using demographic, statistical, user behavioral, and survey based information. For example, subjects are required to complete surveys after exposure to programs and/or commercials. However, survey results often provide only limited information about program and commercial response. For example, survey subjects may be unable or unwilling to express their true thoughts and feelings about a topic, or questions may be phrased with built in bias. Articulate subjects may be given more weight than non-expressive ones. Analysis of multiple survey responses and correlation of the responses to stimulus material is also limited. A variety of semantic, syntactic, metaphorical, cultural, social and interpretive biases and errors prevent accurate and repeatable evaluation. Mechanisms for storing, managing, and retrieving conventional responses are also limited.

Consequently, the techniques and mechanisms of the present invention use neuro-response measurements such as central nervous system, autonomic nervous system, and effector measurements to improve distributed stimulus response data collection and analysis. Data collection mechanisms such as portable EEG, compact video and audio recorders, sensors, etc., are provided to subjects who use the data collection mechanisms in home, work, and recreational environments. In some example, an EEG cap or band may be provided as a baseball cap, hat, headband, or helmet that includes integrated video and audio capture capabilities. EEG dry electrodes monitor neuro-response activity while cameras and microphones monitor eye movement and determine where user attention is focused. For example, cameras may detect that a subject is focused on a billboard or playing a video game. Timing information or other markers may be used to correlate subject activity with neuro-response activity. In particular examples, video and neuro-response data are timestamped or labeled to allow data analysis to later determine stimulus triggers that cause particular EEG spikes.

According to various embodiments, a subject may wear a portable neuro-response data collection mechanism only when a subject is watching television or using a computer. In other examples, the subject may wear the portable neuro-response data collection mechanism during a variety of activities in non-laboratory settings. This allows collection of data from a variety of sources while a subject is in a natural state. In particular embodiments, data collection can occur effectively in corporate and laboratory settings, but it is recognized that neuro-response data may even be more accurate if collected while a subject is in a more natural environment.

According to various embodiments, a portable neuro-response data collection mechanism includes EEG dry electrodes, EOG shielded electrodes for monitoring eye movements, a camera for monitoring whether a subject's attention is focused, a microphone for detecting audio exposure, and another camera for monitoring pupillary dilation. It should be recognized that not all of these elements may be required in a distributed neuro-response data collection mechanism. For example, no pupillary dilation or EOG electrodes may be used. In another example, an eye tracking camera can be used in place of EOG electrodes to monitor both eye movements and pupillary dilation. In particular embodiments, the distributed neuro-response data collection mechanism also includes a wireless transmitter or an interface port for transmitting data. In some examples, the data is transmitted to a computer wirelessly or over a wired interface and automatically provided for data analysis over one or more networks. The data may be processed or filtered before it is provided to a neuro-response data analyzer. In other examples, the data is continuously transmitted over a wireless broadband network.

In particular examples, no video and audio capture capabilities may be included and the neuro-response data collection is correlated with user stated activities such as watching a television program or viewing a commercial. Timing information can be used to correlate neuro-response data with particular television programs and commercials.

A variety of neurological, neuro-physiological, and effector mechanisms may be integrated in a distributed neuro-response data collection mechanism. EEG measures electrical activity associated with post synaptic currents occurring in the milliseconds range. Subcranial EEG can measure electrical activity with the most accuracy, as the bone and dermal layers weaken transmission of a wide range of frequencies. Nonetheless, surface EEG provides a wealth of electrophysiological information if analyzed properly. Portable EEG with dry electrodes provide a large amount of neuro-response information. Electrooculography (EOG), eye tracking, facial emotion encoding, reaction time, etc. can all be measured using small cameras, shielded electrodes, etc.

Some other mechanisms are not yet practical for distributed neuro-response data collection. Mechanisms such as Functional Magnetic Resonance Imaging (fMRI) and Magnetoencephalography (MEG) are not currently portable, although future implementations may render such mechanisms practical. fMRI measures blood oxygenation in the brain that correlates with increased neural activity. According to various embodiments, the techniques and mechanisms of the present invention intelligently blend multiple modes and manifestations of precognitive neural signatures with cognitive neural signatures and post cognitive neurophysiological manifestations to more accurately allow monitoring in distributed environments. In some examples, autonomic nervous system measures are themselves used to validate central nervous system measures. Effector and behavior responses are blended and combined with other measures. According to various embodiments, central nervous system, autonomic nervous system, and effector system measurements are aggregated into a measurement that allows definitive evaluation of subject responses.

According to various embodiments, subjects may be exposed to predetermined or preselected stimulus material. In other examples, no predetermined or preselected stimulus material is provided and a system collects neuro-response data for stimulus material a user is exposed to during typical activities.

For example, multiple subjects may be provided with portable EEG monitoring systems with dry electrodes that allow monitoring of neuro-response activity while subjects run errands or view billboards. Response data is analyzed and integrated. In some examples, all response data is provided for data analysis. In other examples, interesting response data along with recorded stimulus material is provided to a data analyzer. According to various embodiments, response data is analyzed and enhanced for each subject and further analyzed and enhanced by integrating data across multiple subjects.

According to various embodiments, individual and integrated response data is numerically maintained or graphically represented. Measurements for multiple subjects are analyzed to determine possible patterns, fluctuations, profiles, etc.

According to various embodiments, distributed neuro-response data may show particular effectiveness of stimulus material for a particular subset of individuals. A variety of stimulus materials such as entertainment and marketing materials, media streams, billboards, print advertisements, text streams, music, performances, sensory experiences, etc. can be analyzed. According to various embodiments, enhanced neuro-response data is generated using a data analyzer that performs both intra-modality measurement enhancements and cross-modality measurement enhancements. According to various embodiments, brain activity is measured not just to determine the regions of activity, but to determine interactions and types of interactions between various regions. The techniques and mechanisms of the present invention recognize that interactions between neural regions support orchestrated and organized behavior. Attention, emotion, memory, retention, priming, and other characteristics are not merely based on one part of the brain but instead rely on network interactions between brain regions.

The techniques and mechanisms of the present invention further recognize that different frequency bands used for multi-regional communication can be indicative of the effectiveness of stimuli. In particular embodiments, evaluations are calibrated to each subject and synchronized across subjects. In particular embodiments, templates are created for subjects to create a baseline for measuring pre and post stimulus differentials. According to various embodiments, stimulus generators are intelligent and adaptively modify specific parameters such as exposure length and duration for each subject being analyzed.

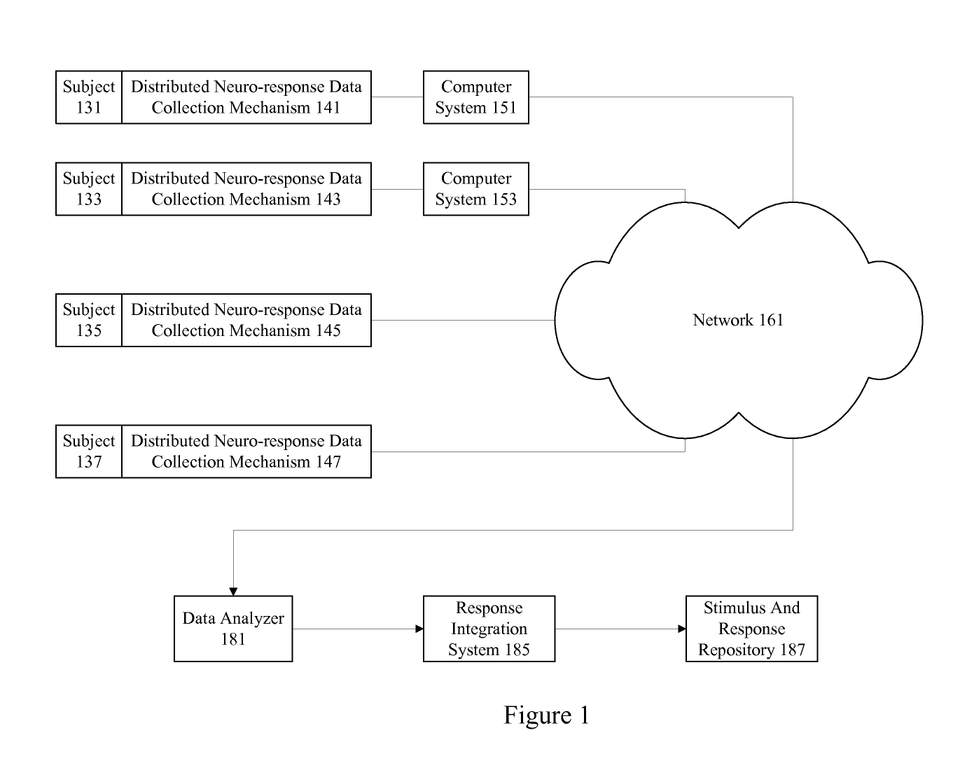

FIG. 1 illustrates one example of a system for distributed collection of neuro-response data. Subjects 131, 133, 135, and 137 are associated with distributed neuro-response data collection mechanisms 141, 143, 145, and 147. According to various embodiments, subjects voluntarily use neuro-response data collection mechanisms such as EEG caps, EOG sensors, recorders, cameras, etc., during exposure to particular stimulus materials or during normal activities in non-laboratory environments. According to various embodiments, neuro-response data is is measured for subjects in non-laboratory settings including homes, shops, workplaces, parks, theatres, etc. In particular embodiments, distributed neuro-response data collection mechanisms 145 and 147 include persistent storage mechanisms and network interfaces that are used to transmit collected data to a data analyzer 181. In other examples, distributed neuro-response data collection mechanisms 141 and 143 include interfaces to computer systems 151 and 153 that are configured to transmit data to a data analyzer 181 over one or more networks.

Materials eliciting neuro-responses from subjects 131, 133, 135, and 137 may include people, activities, brand images, information, performances, entertainment, advertising, and may involve particular tastes, smells, sights, textures and/or sounds. In some examples, stimulus material is selected for presentation to subjects 131, 133, 135, and 137. In other examples, stimulus material subjects are exposed to during normal everyday activities such as driving to work or going to the grocery store are analyzed. Continuous and discrete modes are supported.

According to various embodiments, the subjects 131, 133, 135, and 137 are connected to distributed neuro-response data collection mechanisms 141, 143, 145, and 147. The data collection mechanisms 105 may include a variety of neuro-response measurement mechanisms including neurological and neurophysiological measurements systems such as EEG, EOG, GSR, EKG, pupillary dilation, eye tracking, facial emotion encoding, and reaction time devices, etc. According to various embodiments, neuro-response data includes central nervous system, autonomic nervous system, and/or effector data.

The distributed neuro-response data collection mechanisms 141, 143, 145, and 147 collect neuro-response data from multiple sources. According to various embodiments, data collection mechanisms include central nervous system sources (EEG), autonomic nervous system sources (EKG, pupillary dilation), and effector sources (EOG, eye tracking, facial emotion encoding, reaction time). In particular embodiments, data collected is digitally sampled and stored for later analysis. In particular embodiments, the data collected could be analyzed in real-time. According to particular embodiments, the digital sampling rates are adaptively chosen based on the neurophysiological and neurological data being measured.

In one particular embodiment, the distributed neuro-response data collection mechanism includes EEG 111 measurements made using scalp level electrodes, EOG 113 measurements made using shielded electrodes to track eye data, and a facial affect graphic and video analyzer adaptively derived for each individual.

In particular embodiments, the data collection mechanism are clock synchronized with an image sensor and a recorder. In particular embodiments, the data collection mechanisms 105 also include a condition evaluation subsystem that provides auto triggers, alerts and status monitoring and visualization components that continuously monitor the status of the subject, the direction of attention, stimulus being presented, data being collected, and the data collection instruments. For example, the data collection mechanisms may record neuro-response data while a recorder determines that a subject is listening to a particular song.

The condition evaluation subsystem may also present visual alerts and automatically trigger remedial actions. According to various embodiments, the data collection devices include mechanisms for not only monitoring subject neuro-response to stimulus materials, but also include mechanisms for identifying and monitoring the stimulus materials. For example, data collection mechanisms 105 may be synchronized with a set-top box to monitor channel changes. In other examples, data collection mechanisms 105 may be directionally synchronized to monitor when a subject is no longer paying attention to stimulus material. In still other examples, the data collection mechanisms 105 may receive and store stimulus material generally being viewed by the subject, whether the stimulus is a program, a commercial, printed material, or a scene outside a window of a living room. The data collected allows analysis of neuro-response information and correlation of the information to actual stimulus material and not mere subject distractions.

According to various embodiments, the distributed neuro-response collection system also includes a data cleanser. In particular embodiments, the data cleanser device filters the collected data to remove noise, artifacts, and other irrelevant data using fixed and adaptive filtering, weighted averaging, advanced component extraction (like PCA, ICA), vector and component separation methods, etc. This device cleanses the data by removing both exogenous noise (where the source is outside the physiology of the subject, e.g. a phone ringing while a subject is viewing a video) and endogenous artifacts (where the source could be neurophysiological, e.g. muscle movements, eye blinks, etc.).

The artifact removal subsystem includes mechanisms to selectively isolate and review the response data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (for example, an EEG nearest neighbor weighted averaging approach).

According to various embodiments, the data cleanser device is implemented using hardware, firmware, and/or software and may be integrated into EEG headsets, computer systems, or data analyzers. It should be noted that although a data cleanser device may have a location and functionality that varies based on system implementation.

The data cleanser can pass data to the data analyzer 181. The data analyzer 181 uses a variety of mechanisms to analyze underlying data in the system to determine neuro-response characteristics associated with corresponding stimulus material. According to various embodiments, the data analyzer customizes and extracts the independent neurological and neuro-physiological parameters for each individual in each modality, and blends the estimates within a modality as well as across modalities to elicit an enhanced response to the stimulus material. In some examples, stimulus material recorded using images, video, or audio is synchronized with neuro-response data. In particular embodiments, the data analyzer 181 aggregates the response measures across subjects in a dataset.

According to various embodiments, neurological and neuro-physiological signatures are measured using time domain analyses and frequency domain analyses. Such analyses use parameters that are common across individuals as well as parameters that are unique to each individual. The analyses could also include statistical parameter extraction and fuzzy logic based attribute estimation from both the time and frequency components of the synthesized response.

In some examples, statistical parameters used in a blended effectiveness estimate include evaluations of skew, peaks, first and second moments, population distribution, as well as fuzzy estimates of attention, emotional engagement and memory retention responses.

According to various embodiments, the data analyzer 181 may include an intra-modality response synthesizer and a cross-modality response synthesizer. In particular embodiments, the intra-modality response synthesizer is configured to customize and extract the independent neurological and neurophysiological parameters for each individual in each modality and blend the estimates within a modality analytically to elicit an enhanced response to the presented stimuli. In particular embodiments, the intra-modality response synthesizer also aggregates data from different subjects in a dataset.

According to various embodiments, the cross-modality response synthesizer or fusion device blends different intra-modality responses, including raw signals and signals output. The combination of signals enhances the measures of effectiveness within a modality. The cross-modality response fusion device can also aggregate data from different subjects in a dataset.

According to various embodiments, the data analyzer 181 also includes a composite enhanced effectiveness estimator (CEEE) that combines the enhanced responses and estimates from each modality to provide a blended estimate of the effectiveness. In particular embodiments, blended estimates are provided for each exposure of a subject to stimulus materials. According to various embodiments, numerical values are assigned to each blended estimate. The numerical values may correspond to the intensity of neuro-response measurements, the significance of peaks, the change between peaks, etc. Higher numerical values may correspond to higher significance in neuro-response intensity. Lower numerical values may correspond to lower significance or even insignificant neuro-response activity. In other examples, multiple values are assigned to each blended estimate. In still other examples, blended estimates of neuro-response significance are graphically represented to show changes after repeated exposure.

According to various embodiments, the data analyzer 181 provides analyzed and enhanced response data to a response integration system 185. According to various embodiments, the response integration system 185 combines analyzed and enhanced responses to the stimulus material while using information about stimulus material attributes. In particular embodiments, the response integration system 185 also collects and integrates user behavioral and survey responses with the analyzed and enhanced response data to more effectively measure and neuro-response data collected in a distributed environment.

According to various embodiments, the response integration system 185 obtains characteristics of stimulus material such as requirements and purposes of the stimulus material. Some of these requirements and purposes may be obtained from a stimulus attribute repository. Others may be obtained from other sources. Characteristics may include views and presentation specific attributes such as audio, video, imagery and messages needed, media for enhanced, media for avoidance, etc.

According to various embodiments, the response integration system 185 also includes mechanisms for the collection and storage of demographic, statistical and/or survey based responses to different entertainment, marketing, advertising and other audio/visual/tactile/olfactory material. If this information is stored externally, the response integration system 185 can include a mechanism for the push and/or pull integration of the data, such as querying, extraction, recording, modification, and/or updating.

According to various embodiments, the response integration system 185 integrates the requirements for the presented material, the assessed neuro-physiological and neuro-behavioral response measures, and the additional stimulus attributes such as demographic/statistical/survey based responses into a synthesized measure for various stimulus material consumed by users in various environments.

According to various embodiments, the response integration system 185 provides stimulus and response repository 187 with data including integrated and/or individual stimulus material responses, stimulus attributes, synthesized measures, stimulus material, etc. A variety of data can be stored for later analysis, management, manipulation, and retrieval. In particular embodiments, the repository 187 could be used for tracking stimulus attributes and presentation attributes, audience responses and optionally could also be used to integrate audience measurement information.

According to various embodiments, the information stored in the repository system 187 could be used to assess the audience response to programs/advertisements in multiple regions, across multiple demographics and multiple time spans (days, weeks, months, years, etc.), determine the effectiveness of billboards, monitor neuro-responses to video games and entertainment, etc.

As with a variety of the components in the distributed neuro-response collection system, the response integration system can be co-located with the rest of the system and the user, or could be implemented in a remote location. It could also be optionally separated into an assessment repository system that could be centralized or distributed at the provider or providers of the stimulus material. In other examples, the response integration system is housed at the facilities of a third party service provider accessible by stimulus material providers and/or users.

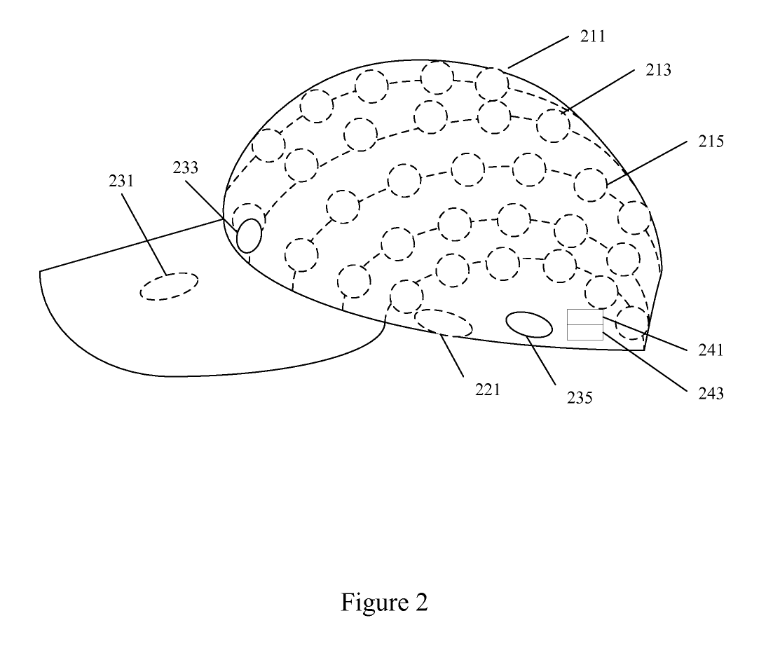

FIG. 2 illustrates a particular example of a distributed neuro-response data collection mechanism. According to various embodiments, the distributed neuro-response data collection mechanism includes multiple EEG dry electrodes including electrodes 211, 213, and 215. In particular embodiments, the EEG dry electrodes operate to detect neurological activity with minimal interference from hair and without use of any electrically conductive gels. According to various embodiments, distributed neuro-response data collection mechanism also includes EOG sensors such as sensor 221 used to detect eye movements. According to various embodiments, a camera 231 is internally oriented and used to detect subject eye movements as well as pupillary dilation. In particular embodiments, the camera 231 is a relatively low resolution camera. In other examples, camera 231 can detect infrared to measure user temperature.

In particular embodiments, another camera 233 is externally oriented and used to determine where user attention is directed. According to various embodiments, the camera 233 records entities in the frame of view of a user. For example, the user may be looking at a menu. The camera 233 would record a video or an image of the menu. In some examples, the externally oriented camera is mounted on a cap itself while an internally oriented camera is mounted on the underside of a bill of a cap. The cameras may obtain media data by taking still pictures, recording video, or maintaining a combination of images and video.. For example, camera 233 may be used to determine that a user is watching a particular commercial or reading a particular book. In some examples, the cameras may may be triggered by EEG neurological activity. According to various embodiments, an audio recorder 235 is provided to detect and/or record audio a subject is exposed to. It should be noted that other sensors and detectors may be included as well. A variety of arrangements are possible.

The data collection mechanism may also include a transmitter and/or receiver to send collected neuro-response data to an data analysis system. In some examples, a transceiver 241 transmits all collected media such as video and/or audio, neuro-response, and sensor data to a data analyzer. In other examples, a transceiver 241 transmits only interested data provided by a filter 243. In other examples, the transceiver 241 also receives information that can be provided to a user or used to modify a system. The filter 243 can remove noise as well as uninteresting portions of collected data. The filter 243 can significantly reduce network usage and can be valuable when limited network resources are available. In some examples, the transceiver can be connected to a computer system that then transmits data over a wide area network to a data analyzer. In other examples, the transceiver sends data over a wide area network to a data analyzer. Other components such as fMRI and MEG that are not yet portable but may become portable at some point may also be integrated into a headset, cap, band, or individual clips attached to a subject's hair.

The headset, cap, band, clips, etc., may be configured in a very discrete manner, so that usage of the distributed neuro-response data collection mechanism would not be obvious to bystanders. and could be used in non-laboratory settings. They may also be configured as to not interfere with most everyday activities.

It should be noted that some components of a neuro-response data collection mechanism have not been shown for clarity. For example, a battery may be required to power components such as cameras and sensors and wiring from the battery are not shown. Similarly, a transceiver may include an antenna that is similarly not shown for clarity purposes. It should also be noted that some components are also optional. For example, multiple cameras may not be required, or EEG electrodes may not be needed if portable fMRI or MEG or other optical imaging mechanisms are available.

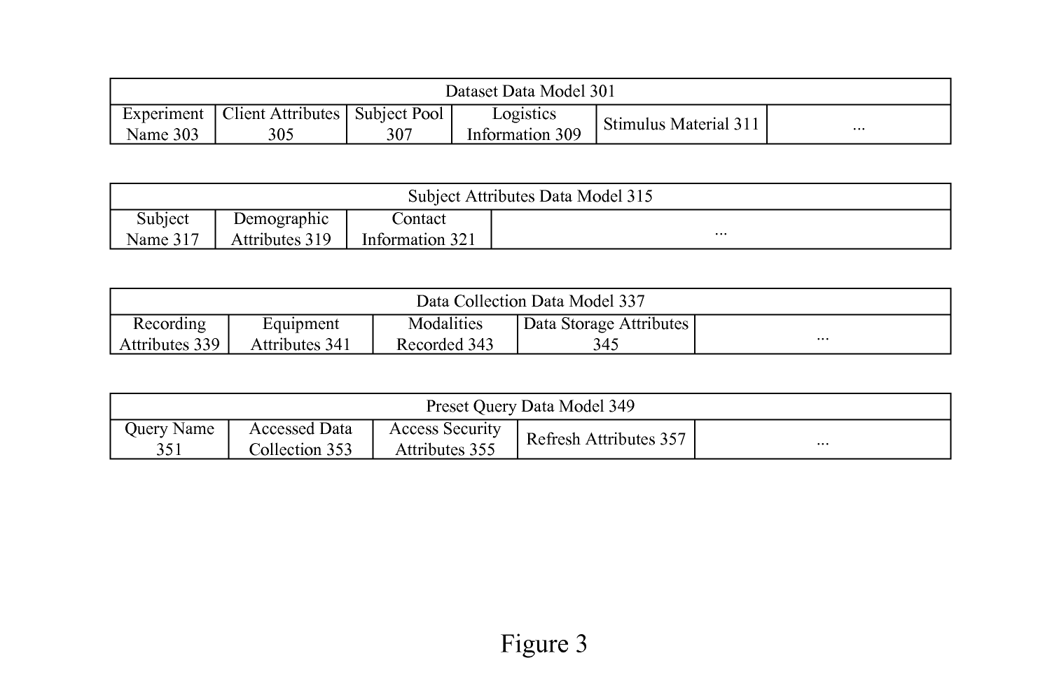

FIG. 3 illustrates examples of data models that can be used for storage of information associated with collection of distributed neuro-response data. According to various embodiments, a dataset data model 301 includes a name 303 and/or identifier, client attributes 305, a subject pool 307, logistics information 309 such as the location, date, and stimulus material 311 identified using user entered information or video and audio detection.

In particular embodiments, a subject attribute data model 315 includes a subject name 317 and/or identifier, contact information 321, and demographic attributes 319 that may be useful for review of neurological and neuro-physiological data. Some examples of pertinent demographic attributes include marriage status, employment status, occupation, household income, household size and composition, ethnicity, geographic location, sex, race. Other fields that may be included in data model 315 include shopping preferences, entertainment preferences, and financial preferences. Shopping preferences include favorite stores, shopping frequency, categories shopped, favorite brands. Entertainment preferences include network/cable/satellite access capabilities, favorite shows, favorite genres, and favorite actors. Financial preferences include favorite insurance companies, preferred investment practices, banking preferences, and favorite online financial instruments. A variety of subject attributes may be included in a subject attributes data model 315 and data models may be preset or custom generated to suit particular purposes.

Other data models may include a data collection data model 337. According to various embodiments, the data collection data model 337 includes recording attributes 339, equipment identifiers 341, modalities recorded 343, and data storage attributes 345. In particular embodiments, equipment attributes 341 include an amplifier identifier and a sensor identifier.

Modalities recorded 343 may include modality specific attributes like EEG cap layout, active channels, sampling frequency, and filters used. EOG specific attributes include the number and type of sensors used, location of sensors applied, etc. Eye tracking specific attributes include the type of tracker used, data recording frequency, data being recorded, recording format, etc. According to various embodiments, data storage attributes 345 include file storage conventions (format, naming convention, dating convention), storage location, archival attributes, expiry attributes, etc.

A preset query data model 349 includes a query name 351 and/or identifier, an accessed data collection 353 such as data segments involved (models, databases/cubes, tables, etc.), access security attributes 355 included who has what type of access, and refresh attributes 357 such as the expiry of the query, refresh frequency, etc. Other fields such as push-pull preferences can also be included to identify an auto push reporting driver or a user driven report retrieval system.

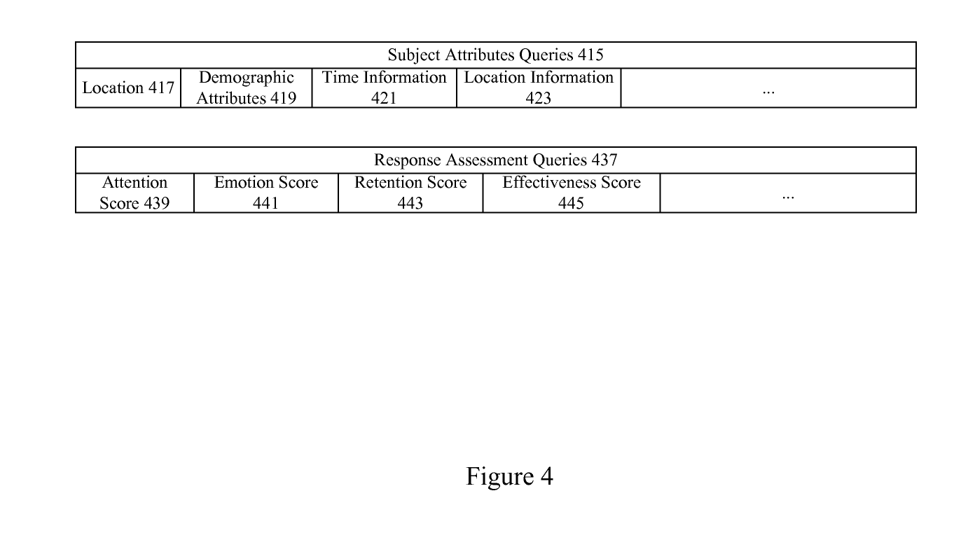

FIG. 4 illustrates examples of queries that can be performed to obtain data associated with distributed neuro-response data collection. According to various embodiments, queries are defined from general or customized scripting languages and constructs, visual mechanisms, a library of preset queries, diagnostic querying including drill-down diagnostics, and eliciting what if scenarios. According to various embodiments, subject attributes queries 415 may be configured to obtain data from a neuro-informatics repository using a location 417 or geographic information, session information 421 such as timing information for the data collected. Location information 423 may also be collected. In some examples, a distributed neuro-response data collection mechanism includes GPS or other location detection mechanisms. Demographics attributes 419 include household income, household size and status, education level, age of kids, etc.

Other queries may retrieve stimulus material recorded based on shopping preferences of subject participants, countenance, physiological assessment, completion status. For example, a user may query for data associated with product categories, products shopped, shops frequented, subject eye correction status, color blindness, subject state, signal strength of measured responses, alpha frequency band ringers, muscle movement assessments, segments completed, etc.

Response assessment based queries 437 may include attention scores 439, emotion scores, 441, retention scores 443, and effectiveness scores 445. Such queries may obtain materials that elicited particular scores. Response measure profile based queries may use mean measure thresholds, variance measures, number of peaks detected, etc. Group response queries may include group statistics like mean, variance, kurtosis, p-value, etc., group size, and outlier assessment measures. Still other queries may involve testing attributes like test location, time period, test repetition count, test station, and test operator fields. A variety of types and combinations of types of queries can be used to efficiently extract data.

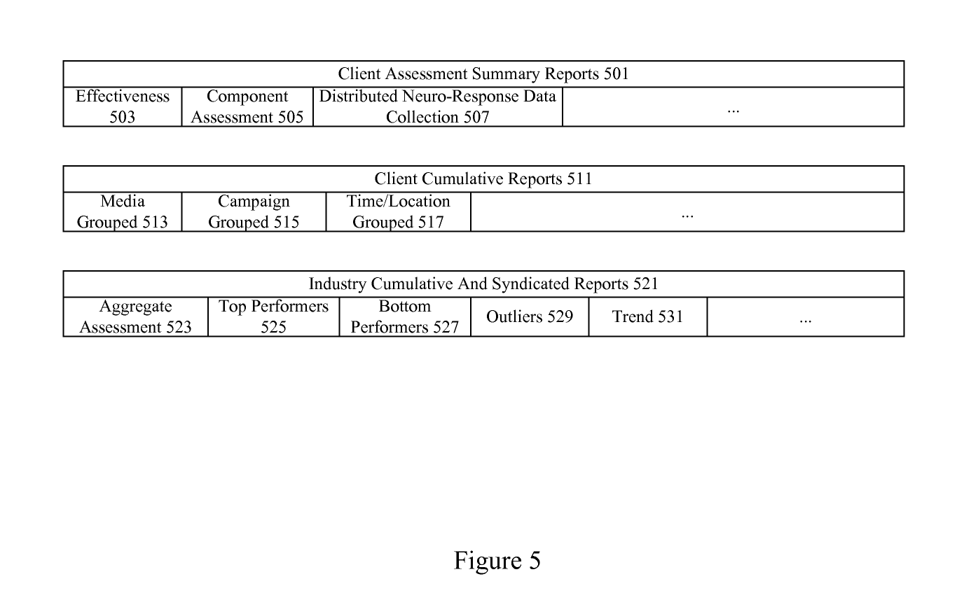

FIG. 5 illustrates examples of reports that can be generated. According to various embodiments, client assessment summary reports 501 include effectiveness measures 503, component assessment measures 505, and distributed neuro-response data collection measures 507. Effectiveness assessment measures include composite assessment measure(s), industry/category/client specific placement (percentile, ranking, etc.), actionable grouping assessment such as removing material, modifying segments, or fine tuning specific elements, etc, and the evolution of the effectiveness profile over time. In particular embodiments, component assessment reports include component assessment measures like attention, emotional engagement scores, percentile placement, ranking, etc. Component profile measures include time based evolution of the component measures and profile statistical assessments. According to various embodiments, reports include the number of times material is assessed, attributes of the multiple presentations used, evolution of the response assessment measures over the multiple presentations, and usage recommendations.

According to various embodiments, client cumulative reports 511 include media grouped reporting 513 of all stimulus assessed, campaign grouped reporting 515 of stimulus assessed, and time/location grouped reporting 517 of stimulus assessed. According to various embodiments, industry cumulative and syndicated reports 521 include aggregate assessment responses measures 523, top performer lists 525, bottom performer lists 527, outliers 529, and trend reporting 531. In particular embodiments, tracking and reporting includes specific products, categories, companies, brands.

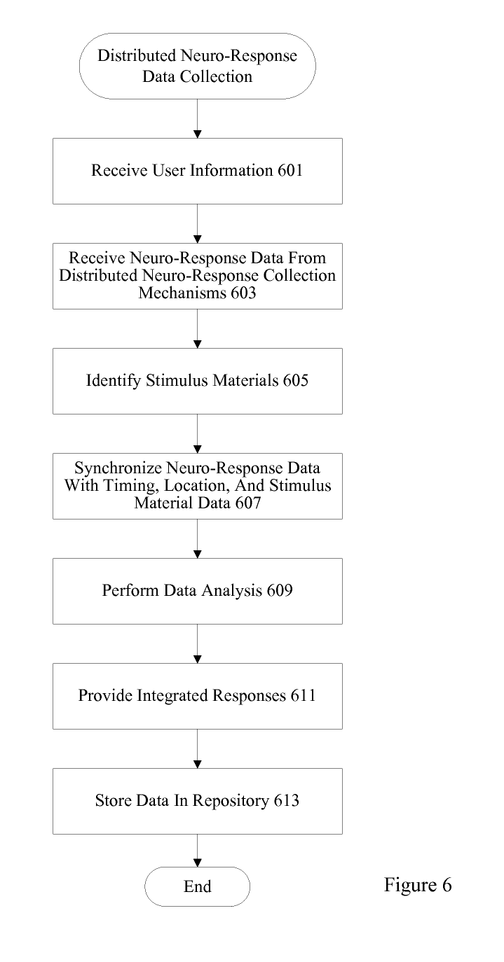

FIG. 6 illustrates one example of distributed neuro-response data collection. At 601, user information is received from a subject provided with a neuro-response data collection mechanism. According to various embodiments, the subject sends data including age, gender, income, location, interest, ethnicity, etc. after being provided with an EEG cap including EEG electrodes, EOG sensors, cameras, recorders, network interfaces, and a global position system (GPS) device integrated into an unobtrusive device that can be worn during typical activities.

At 603, neuro-response data, video and audio recorded data, timing information, and/or location information, etc., is received from the subject neuro-response data collection mechanism. According to various embodiments, EEG, EOG, pupillary dilation, facial emotion encoding data, video, images, audio, GPS data, timestamps, etc., are transmitted from the subject to a neuro-response data analyzer. In particular embodiments, data is filtered and compressed prior to transmission. For example, only video and audio corresponding to neuro-logically salient events are transmitted to save on network bandwidth. According to various embodiments, neuro-response and associated data is transmitted directly from an EEG cap wide area network interface to a data analyzer. In particular embodiments, neuro-response and associated data is transmitted to a computer system that then performs compression and filtering of the data before transmitting the data to a data analyzer over a network.

According to various embodiments, data is also passed through a data cleanser to remove noise and artifacts that may make data more difficult to interpret. According to various embodiments, the data cleanser removes EEG electrical activity associated with blinking and other endogenous/exogenous artifacts. Data cleansing may be performed before or after data transmission to a data analyzer.

At 605, stimulus material is identified. According to various embodiments, stimulus material is identified based on user input. For example, a user watching a particular movie may enter the title of the movie along with how and where it was viewed. Alternatively, video recording may be analyzed using text, facial, brand, video, image, and audio recognition algorithms to determine what the user was viewing. Eye tracking movements can determine where user attention is focused at any given time. Although that eye movements do occur when attention is diverted, it is recognized that focused attention typically occurs when eye position is focused in the forward direction. Consequently, the EEG cap and video camera direction typically coincide with the direction of user attention. EEG data may also be tagged to indicate correspondence with particular video and audio events. According to various embodiments, a user walking down a supermarket aisle may direct attention to certain products that are identified using video recordings and correlated with neuro-response measures to determine the effectiveness of product labeling.

At 607, neuro-response data is synchronized with timing, location, and other stimulus material data. According to various embodiments, neuro-response data such as EEG and EOG data is tagged to indicate what the subject is viewing or listening to at a particular time.

At 609, data analysis is performed. Data analysis may include intra-modality response synthesis and cross-modality response synthesis to enhance effectiveness measures. It should be noted that in some particular instances, one type of synthesis may be performed without performing other types of synthesis. For example, cross-modality response synthesis may be performed with or without intra-modality synthesis.

A variety of mechanisms can be used to perform data analysis 609. In particular embodiments, a stimulus attributes repository is accessed to obtain attributes and characteristics of the stimulus materials, along with purposes, intents, objectives, etc. In particular embodiments, EEG response data is synthesized to provide an enhanced assessment of effectiveness. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making Gamma waves occur between 30 and 60 Hz and are involved in binding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment.

However, the techniques and mechanisms of the present invention recognize that analyzing high gamma band (kappa-band: Above 60 Hz) measurements, in addition to theta, alpha, beta, and low gamma band measurements, enhances neurological attention, emotional engagement and retention component estimates. In particular embodiments, EEG measurements including difficult to detect high gamma or kappa band measurements are obtained, enhanced, and evaluated. Subject and task specific signature sub-bands in the theta, alpha, beta, gamma and kappa bands are identified to provide enhanced response estimates. According to various embodiments, high gamma waves (kappa-band) above 80 Hz (typically detectable with sub-cranial EEG and/or magnetoencephalograophy) can be used in inverse model-based enhancement of the frequency responses to the stimuli.

Various embodiments of the present invention recognize that particular sub-bands within each frequency range have particular prominence during certain activities. A subset of the frequencies in a particular band is referred to herein as a sub-band. For example, a sub-band may include the 40-45 Hz range within the gamma band. In particular embodiments, multiple sub-bands within the different bands are selected while remaining frequencies are band pass filtered. In particular embodiments, multiple sub-band responses may be enhanced, while the remaining frequency responses may be attenuated.

An information theory based band-weighting model is used for adaptive extraction of selective dataset specific, subject specific, task specific bands to enhance the effectiveness measure. Adaptive extraction may be performed using fuzzy scaling. Stimuli can be presented and enhanced measurements determined multiple times to determine the variation profiles across multiple presentations. Determining various profiles provides an enhanced assessment of the primary responses as well as the longevity (wear-out) of the marketing and entertainment stimuli. The synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

Although a variety of synthesis mechanisms are described, it should be recognized that any number of mechanisms can be applied—in sequence or in parallel with or without interaction between the mechanisms.

Although intra-modality synthesis mechanisms provide enhanced significance data, additional cross-modality synthesis mechanisms can also be applied. A variety of mechanisms such as EEG, Eye Tracking, GSR, EOG, and facial emotion encoding are connected to a cross-modality synthesis mechanism. Other mechanisms as well as variations and enhancements on existing mechanisms may also be included. According to various embodiments, data from a specific modality can be enhanced using data from one or more other modalities. In particular embodiments, EEG typically makes frequency measurements in different bands like alpha, beta and gamma to provide estimates of significance. However, the techniques of the present invention recognize that significance measures can be enhanced further using information from other modalities.

For example, facial emotion encoding measures can be used to enhance the valence of the EEG emotional engagement measure. EOG and eye tracking saccadic measures of object entities can be used to enhance the EEG estimates of significance including but not limited to attention, emotional engagement, and memory retention. According to various embodiments, a cross-modality synthesis mechanism performs time and phase shifting of data to allow data from different modalities to align. In some examples, it is recognized that an EEG response will often occur hundreds of milliseconds before a facial emotion measurement changes. Correlations can be drawn and time and phase shifts made on an individual as well as a group basis. In other examples, saccadic eye movements may be determined as occurring before and after particular EEG responses. According to various embodiments, time corrected GSR measures are used to scale and enhance the EEG estimates of significance including attention, emotional engagement and memory retention measures.

Evidence of the occurrence or non-occurrence of specific time domain difference event-related potential components (like the DERP) in specific regions correlates with subject responsiveness to specific stimulus. According to various embodiments, ERP measures are enhanced using EEG time-frequency measures (ERPSP) in response to the presentation of the marketing and entertainment stimuli. Specific portions are extracted and isolated to identify ERP, DERP and ERPSP analyses to perform. In particular embodiments, an EEG frequency estimation of attention, emotion and memory retention (ERPSP) is used as a co-factor in enhancing the ERP, DERP and time-domain response analysis.

EOG measures saccades to determine the presence of attention to specific objects of stimulus. Eye tracking measures the subject's gaze path, location and dwell on specific objects of stimulus. According to various embodiments, EOG and eye tracking is enhanced by measuring the presence of lambda waves (a neurophysiological index of saccade effectiveness) in the ongoing EEG in the occipital and extra striate regions, triggered by the slope of saccade-onset to estimate the significance of the EOG and eye tracking measures. In particular embodiments, specific EEG signatures of activity such as slow potential shifts and measures of coherence in time-frequency responses at the Frontal Eye Field (FEF) regions that preceded saccade-onset are measured to enhance the effectiveness of the saccadic activity data.

According to various embodiments, facial emotion encoding uses templates generated by measuring facial muscle positions and movements of individuals expressing various emotions prior to the testing session. These individual specific facial emotion encoding templates are matched with the individual responses to identify subject emotional response. In particular embodiments, these facial emotion encoding measurements are enhanced by evaluating inter-hemispherical asymmetries in EEG responses in specific frequency bands and measuring frequency band interactions. The techniques of the present invention recognize that not only are particular frequency bands significant in EEG responses, but particular frequency bands used for communication between particular areas of the brain are significant. Consequently, these EEG responses enhance the EMG, graphic and video based facial emotion identification.

Integrated responses are generated at 611. According to various embodiments, the data communication device transmits data to the response integration using protocols such as the File Transfer Protocol (FTP), Hypertext Transfer Protocol (HTTP) along with a variety of conventional, bus, wired network, wireless network, satellite, and proprietary communication protocols. The data transmitted can include the data in its entirety, excerpts of data, converted data, and/or elicited response measures. According to various embodiments, data is sent using a telecommunications, wireless, Internet, satellite, or any other communication mechanisms that is capable of conveying information from multiple subject locations for data integration and analysis. The mechanism may be integrated in a set top box, computer system, receiver, mobile device, etc.

In particular embodiments, the data communication device sends data to the response integration system. According to various embodiments, the response integration system combines analyzed and enhanced responses to the stimulus material while using information about stimulus material attributes. In particular embodiments, the response integration system also collects and integrates user behavioral and survey responses with the analyzed and enhanced response data to more effectively measure and track distributed neuro-responses to stimulus materials. According to various embodiments, the response integration system obtains attributes such as requirements and purposes of the stimulus material presented.

Some of these requirements and purposes may be obtained from a variety of databases. According to various embodiments, the response integration system also includes mechanisms for the collection and storage of demographic, statistical and/or survey based responses to different entertainment, marketing, advertising and other audio/visual/tactile/olfactory material. If this information is stored externally, the response integration system can include a mechanism for the push and/or pull integration of the data, such as querying, extraction, recording, modification, and/or updating.

The response integration system can further include an adaptive learning component that refines user or group profiles and tracks variations in the distributed neuro-response data collection system to particular stimuli or series of stimuli over time. This information can be made available for other purposes, such as use of the information for presentation attribute decision making. According to various embodiments, the response integration system builds and uses responses of users having similar profiles and demographics to provide integrated responses at 611. In particular embodiments, stimulus and response data is stored in a repository at 613 for later retrieval and analysis.

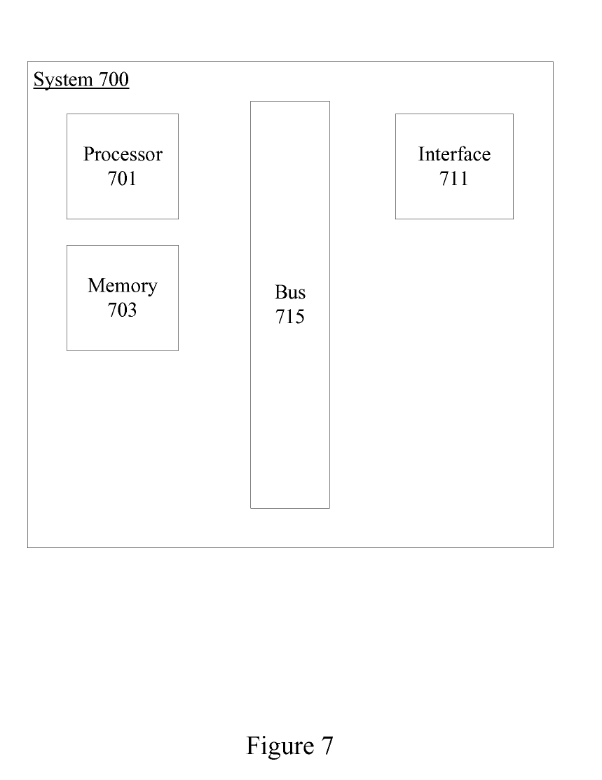

According to various embodiments, various mechanisms such as the data collection mechanisms, the intra-modality synthesis mechanisms, cross-modality synthesis mechanisms, etc. are implemented on multiple devices. However, it is also possible that the various mechanisms be implemented in hardware, firmware, and/or software in a single system. FIG. 7 provides one example of a system that can be used to implement one or more mechanisms. For example, the system shown in FIG. 7 may be used to implement a data analyzer.

According to particular example embodiments, a system 700 suitable for implementing particular embodiments of the present invention includes a processor 701, a memory 703, an interface 711, and a bus 715 (e.g., a PCI bus). When acting under the control of appropriate software or firmware, the processor 701 is responsible for such tasks such as pattern generation. Various specially configured devices can also be used in place of a processor 701 or in addition to processor 701. The complete implementation can also be done in custom hardware. The interface 711 is typically configured to send and receive data packets or data segments over a network. Particular examples of interfaces the device supports include host bus adapter (HBA) interfaces, Ethernet interfaces, frame relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as data synthesis.

According to particular example embodiments, the system 700 uses memory 703 to store data, algorithms and program instructions. The program instructions may control the operation of an operating system and/or one or more applications, for example. The memory or memories may also be configured to store received data and process received data.

Because such information and program instructions may be employed to implement the systems/methods described herein, the present invention relates to tangible, machine readable media that include program instructions, state information, etc. for performing various operations described herein. Examples of machine-readable media include, but are not limited to, magnetic media such as hard disks, floppy disks, and magnetic tape; optical media such as CD-ROM disks and DVDs; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

Although the foregoing invention has been described in some detail for purposes of clarity of understanding, it will be apparent that certain changes and modifications may be practiced within the scope of the appended claims. Therefore, the present embodiments are to be considered as illustrative and not restrictive and the invention is not to be limited to the details given herein, but may be modified within the scope and equivalents of the appended claims.

What is claimed is:

1. A method, comprising:
receiving neuro-response data from a plurality of neuro-response data collection mechanisms associated with a plurality of users exposed to marketing and entertainment materials, the neuro-response data collection mechanisms comprising a plurality of electroencephalography (EEG) electrodes, wherein the neuro-response data is collected in a plurality of disparate settings and received over a network;
analyzing the neuro-response data to determine the effectiveness of the marketing and entertainment materials that the plurality of users are exposed to in the plurality of disparate settings;
sending effectiveness data to the plurality of disparate settings.

2. The method of claim 1, wherein analyzing the neuro-response data comprises identifying evidence of the occurrence or non-occurrence of specific time domain difference event-related potential (DERP) components.

3. The method of claim 2, wherein neuro-response data is synchronized with stimulus material.

4. The method of claim 1, wherein neuro-response data is received from distributed neuro-response data collection mechanisms associated with the plurality of users in a plurality of geographic locations.

5. The method of claim 1, wherein the plurality of disparate settings comprise a plurality of corporate settings.

6. The method of claim 1, wherein the plurality of disparate settings comprise a plurality of laboratory settings.

7. The method of claim 1, wherein the neuro-response data collection mechanism further comprises electrooculography (EOG) sensors.

8. The method of claim 1, wherein the plurality of EEG electrodes are EEG dry electrodes.

9. A system, comprising:
an interface configured to receive neuro-response data from a plurality of neuro-response data collection mechanisms associated with a plurality of users exposed to marketing and entertainment materials, the neuro-response data collection mechanism comprising a plurality of electroencephalography (EEG) electrodes, wherein the neuro-response data is collected in a plurality of disparate settings and received over a network;
a processor configured to analyze the neuro-response data to determine the effectiveness of the marketing and entertainment materials that the plurality of users are exposed to in the plurality of disparate settings;
wherein effectiveness data is sent to the plurality of disparate settings.

10. The system of claim 9, wherein analyzing the neuro-response data comprises identifying evidence of the occurrence or non-occurrence of specific time domain difference event-related potential (DERP) components.

11. The system of claim 10, wherein neuro-response data is synchronized with stimulus material.

12. The system of claim 9, wherein neuro-response data is received from distributed neuro-response data collection mechanisms associated with the plurality of users in a plurality of geographic locations.

13. The system of claim 9, wherein the plurality of disparate settings comprise a plurality of corporate settings.

14. The system of claim 9, wherein the plurality of disparate settings comprise a plurality of laboratory settings.

15. The system of claim 9, wherein the neuro-response data collection mechanism further comprises electrooculography (EOG) sensors.

16. The system of claim 9, wherein the plurality of EEG electrodes are EEG dry electrodes.

17. An apparatus, comprising:
means for receiving neuro-response data from a plurality of neuro-response data collection mechanisms associated with a plurality of users exposed to marketing and entertainment materials, the neuro-response data collection mechanism comprising a plurality of electroencephalography (EEG) electrodes, wherein the neuro-response data is collected in a plurality of disparate settings and received over a network;
means for analyzing the neuro-response data to determine the effectiveness of the marketing and entertainment materials that the plurality of users are exposed to in the plurality of disparate settings;
means for sending effectiveness data to the plurality of disparate settings.

18. The apparatus of claim 17, wherein analyzing the neuro-response data comprises identifying evidence of the occurrence or non-occurrence of specific time domain difference event-related potential (DERP) components.

19. The apparatus of claim 18, wherein neuro-response data is synchronized with stimulus material.

20. The apparatus of claim 17, wherein neuro-response data is received from distributed neuro-response data collection mechanisms associated with the plurality of users in a plurality of geographic locations.
